(19) United States
(12) Patent Application Publication
(10) Pub. No.: US 2020/0113498 A1
(43) Pub. Date: Apr. 16, 2020

(54) WIRELESS PULSE OXIMETER DEVICE

(71) Applicant: 3M INNOVATIVE PROPERTIES COMPANY

(72) Inventor: WILLIAM BEDINGHAM, WOODBURY, MN (US)

(73) Assignee: 3M INNOVATIVE PROPERTIES COMPANY, SAINT PAUL, MN (US)

(21) Appl. No.: 16/621,294

(22) PCT Filed: Jun. 13, 2018
(86) PCT No.: PCT/US2018/037204

§ 371 Date: Dec. 11, 2019

Related U.S. Application Data
(60) Provisional application No. 62/524,319, filed on Jun. 23, 2017.

(51) Int. Cl. A61B 5/1455; A61B 5/00

(52) U.S. Cl. CPC A61B 5/14552; A61B 5/0004; A61B 5/02433; A61B 5/742; A61B 5/7278; A61B 5/7203

(57) ABSTRACT

A wireless pulse oximeter device can include a front-end circuit. The device also include a wireless communications module to communicate with a medical monitor or wireless receiver device. The device can also have a controller communicatively coupled to the front-end circuit and the wireless communication module. The controller can have one or more processors configured to receive the at least two photodiode readings, determine an AC component value and a DC component value of a first one of the at least two photodiode readings, transmit the AC component value, determine an R-value corresponding to a ratio of an optical absorption of a first wavelength of light to an optical absorption of a second wavelength of light, for a first set of photodiode readings, and transmit the R-value for the first set of photodiode readings.

### BACKGROUND

Pulse oximetry or SpO2 sensing can be used in the medical setting to monitor a patient's heart rate, oxygen saturation, and pressure photoplethysmogram (PPG). A pulse oximeter device can be attached to the patient body on a fingertip, forehead, earlobe or sternum.

Medical monitors can have a dedicated SpO2 connector that interfaces with a pulse oximeter device. The pulse oximeter device can form a wired connection through a cable which can interface with pins on the medical monitor corresponding to red and infrared (IR) light emitting diodes (LEDs) and a photodiode(PD).

Driving and receiving electronics can be located within the medical monitor, and the optical emission and detection elements are located at the patient interface. Every second, hundreds of red and infrared LED signals (e.g., current pulses) can be transmitted to the LEDs of the pulse oximeter and the resultant photodiode signal current streaming from the photodiode can be measured. The medical monitor can internally processes this information to extract the AC and DC components of each LED signal, an R-ratio, and computes the oxygen saturation from these values.

While this system has been effective, reusable cables can present cleanliness challenges and also hamper patient mobility and comfort. Wireless pulse oximetry systems have been proposed but the data sampling rates and the 16-24 bit fidelity to accurately capture pulse oximeter data can be problematic not only on the sender-side (where battery life and transmissive capabilities hinder the sampling rate and resolution of a PD reading), but also on receiver-side (where medical monitors are configured analyze analog PD readings using internal mechanisms).

For example, the LED and PD signals cannot be easily transmitted back and forth to through a wireless pulse oximetry sensor and receiver without significant effects on the battery life of the sensor. Further, the wireless signals transmitted back and forth need to be precisely synchronized or the system can fail.

### SUMMARY

Aspects of the present disclosure provide for a wireless pulse oximeter device that includes a front-end circuit. The front-end circuit can include an LED driver circuit having at least one LED and configured to provide at least two wavelengths of light. The front-end circuit can include a photodiode configured to provide at least two photodiode readings to the at least two wavelengths of light from the at least one LED, wherein the at least two photodiode readings is indicative of light absorption of arterial blood in a patient at each of the at least two wavelengths of light.

The device can also include a wireless communications module to communicate with a medical monitor or wireless receiver device. The device can also have a controller communicatively coupled to the front-end circuit and the wireless communication module. The controller can have one or more processors configured to receive the at least two photodiode readings, determine an AC component value and a DC component value of a first one of the at least two photodiode readings, transmit the AC component value, determine an R-value corresponding to a ratio of an optical absorption of a first wavelength of light to an optical absorption of a second wavelength of light, for a first set of photodiode readings, and transmit the R-value for the first set of photodiode readings.

### BRIEF DESCRIPTION OF THE DRAWINGS

FIG. 1 illustrates a block diagram of a system of wireless pulse oximetry, according to various aspects of the present disclosure.

FIG. 2 illustrates a block diagram of a wireless pulse oximeter receiver, according to various aspects of the present disclosure.

FIG. 3 illustrates a flowchart of a method of operating the receiver, according to various aspects of the present disclosure.

FIG. 4 illustrates a flowchart of a method of receiving a wireless signal, according to various aspects of the present disclosure.

FIG. 5 illustrates a flowchart of a method of determining a first photodiode signal from a wireless signal based on an LED activation signal, according to various aspects of the present disclosure.

FIG. 6 illustrates an exemplary timing diagram at a medical monitor timing circuit, according to various aspects of the present disclosure.

FIG. 7A illustrates an exemplary high-level timing diagram of a translation circuit, according to various aspects of the present disclosure.

FIG. 7B illustrates an exemplary timing diagram of FIG. 7A in greater detail, according to various aspects of the present disclosure.

FIG. 8 illustrates schematic block diagram of a pulse oximeter device, according to aspects of the present disclosure.

FIG. 9 illustrate a flowchart of a method for the pulse oximeter device, according to aspects of the present disclosure.

FIG. 10 illustrates a flowchart of a method for receiving an photodiode reading, according to aspects of the present disclosure.

FIG. 11 illustrates a flowchart of a method for determining an AC component value and a DC component value for a photodiode reading of a wavelength of light, according to aspects of the present disclosure.

FIG. 12 illustrates a flowchart of a method for determining an R-value of a set of photodiode readings, according to aspects of the present disclosure.

### DETAILED DESCRIPTION

Aspects of the present disclosure relate to a wireless receiver device for receiving a wireless signal from a wireless pulse oximeter that is sufficient to construct a photoplethysmogram. The wireless signal can be a portion of the photoplethysmogram waveform (e.g., at least one AC component value for a particular wavelength of light) and an R-value or measurement derived from an R-value (e.g., SpO2). The wireless receiver device can receive the wireless signal and reconstruct a photoplethysmogram waveform from the portion and the R-value and timed based on one or more light emitting diode (LED) activation signals received from a medical monitor.

Aspects of the present disclosure also provide for a wireless pulse oximeter device configured to transmit an AC component value but not a DC component value corresponding to an LED photodiode response to a first or second LED, and an R-value.

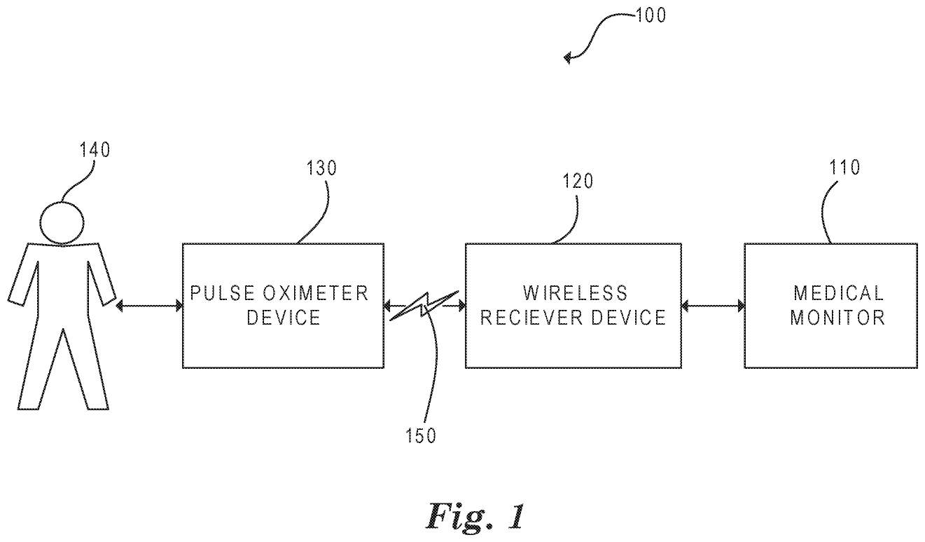

FIG. 1 illustrates an overview of a system 100 for performing wireless pulse oximetry. Some components of the system 100 can be described further herein.

The system 100 can have a medical monitor 110. The medical monitor 110 is a device that displays a photoplethysmogram and displays an blood oxygen saturation level of a patient 140 based on pulse oximetry measurements. The medical monitor 110 is generally remote from the pulse oximeter device 130. The medical monitor 110 can be a multifunctional medical monitor that displays, in addition to the blood saturation level, the EKG, pulse, and blood pressure of the patient 140. Examples of medical monitors are available under the trade designation Intellivue, Model MP70 from Koninklijke Philips N. V., Netherlands or Dash, Model 5000 from General Electric (New York),

In at least one embodiment, the medical monitor 110 is configured to couple to a pulse oximeter using a first wire (e.g., a black wire) to transmit a first analog LED activation signal to activate an first LED on the oximeter and a second wire (e.g., a red wire) to transmit a second analog LED activation signal to activate a second LED on the oximeter. The medical monitor can use a third wire (e.g., a white wire) to receive a photodiode signal and a fourth wire (e.g., a green wire) to use as a ground for the third wire. The medical monitor 110 may have one or more components to process the photodiode signal (received from a directly connected pulse oximeter) and produce a photoplethysmogram and an oxygen saturation value of the patient 140. In at least one embodiment, a separate wireless receiver device 120 may be useful due to being backwards compatible with wired systems such that the medical monitor 110 can receive a wireless signal from a pulse oximeter device 130 without modification of any legacy medical monitors 110.

In at least one embodiment, the medical monitor 110 can be configured to receive wireless digital signals from the pulse oximeter device 130 directly. For example, the medical monitor 110 can have a wireless receiver on-board and perform analysis using raw data sent from a wireless pulse oximeter device 130. In at least one embodiment, the medical monitor 110 can have a receiver circuit for receiving an R-value and an AC component corresponding to one wavelength of light from the pulse oximeter device 130 and determining a photoplethysmogram, peripheral capillary oxygen saturation (SpO2), pulse from only these values. In at least one embodiment, the receiver circuit can receive a complete photodiode signal and display the SpO2 and photoplethysmogram. If the medical monitor 110 is configured to receive the wireless signal 150 directly, then the wireless receiver device 120 can be integrated with the medical monitor 110 (thus, some components of the wireless receiver device 120 can be optional such as the D/A circuit).

The system 100 can include a wireless receiver device 120. In at least one embodiment, the wireless receiver device 120 converts a wireless signal 150 to an output based on a timing pulse from the medical monitor 110.

In at least one embodiment, the wireless receiver device 120 can be separate from the medical monitor 110. The wireless receiver device 120 can be configured to convert a wireless photodiode signal to an photodiode signal timed based on inputs from the medical monitor 110. The photodiode signal can contain data sufficient for the medical monitor 110 to reconstruct a photoplethysmogram.

In at least one embodiment, the wireless receiver device 120 can be integrated with the medical monitor 110 as described above. Generally, the wireless receiver device 120 can perform analysis of the wireless photodiode signal sufficient for the medical monitor 110 to extract a photoplethysmogram and an oxygen saturation value for the patient. In at least one example, the wireless receiver device 120 can receive a wireless signal 150 from a pulse oximeter device 130 and receive a digital or analog LED activation signal from a component within the medical monitor 110. The output to the medical monitor 110 can be digital or, preferably, analog.

The system 100 can include a wireless signal 150. The wireless signal 150 can communicatively couple the pulse oximeter device 130 and the wireless receiver device 120. The wireless signal 150 can be a Radio Frequency (RF) signal.

As used herein, a wireless signal can refer to one or more values (e.g., AC component values, and R-values). The wireless signal can also include a wireless photodiode signal and an R-value signal. The wireless photodiode signal can refer to one or more AC component values for a patient that correspond to one LED. The R-value signal can refer to one or more R-values for a patient which is based on two LEDs.

In at least one embodiment, the wireless signal 150 can be in the form of data packets. For example, data packets may be unpacked or processed by one or more microprocessors on the wireless receiver device 120 or the pulse oximeter device 130. In at least one embodiment, the wireless signal 150 can be sent using a Bluetooth protocol or WiFI using IEEE 802.11 protocols or even ultra-wide band. Preferably, the wireless signal 150 can operate using a medical body area network (MBAN) which can operate in the 2360-2390 MHz band or the 2390-2400 MHz band.

In at least one embodiment, the wireless signal 150 can be bit stream-based. For example a wireless signal 150 can be received bit-by-bit which may improve signal fidelity. In some embodiments, an offset value may be useful where the wireless signal 150 is sent in the raw signal to the wireless receiver device 120.

The system 100 can include a pulse oximeter device 130. The pulse oximeter device 130 can be a device that measures an photoelectrical response to at least one wavelength of light. As used herein, light can refer to electromagnetic radiation that has a wavelength in the range from about 1000000 nm to 10 nm. Some wavelengths of light, e.g., red, may perceived by the unaided, normal human eye. The pulse oximeter device 130 can result in a determination of SpO2 or oxygen saturation level of a patient. In at least one embodiment, the pulse oximeter device 130 can be referred to as a sensor which responds to a physical stimulus and transmits a resulting impulse for interpretation or measurement or for operating a control.

The pulse oximeter device 130 can rely on transmissive or reflected light to determine the photoelectrical response. The pulse oximeter device 130 can be wireless. An aspect of the present disclosure is that the pulse oximeter device 130 performs processing locally such that the pulse oximeter device 130 transmits only a portion of a photoplethysmogram waveform. Although described with greater detail herein, the pulse oximeter device 130 can have at least a first LED configured to emit at least two wavelengths of light, a photodiode, a controller, and a wireless communication module.

The system 100 can also include a patient 140. The patient 140 is generally mammalian, and preferably human. The pulse oximeter device 130 can be positioned over a target tissue. For example, the pulse oximeter device 130 can specifically be removably attached (meaning adhered, mechanically clipped (e.g., using a spring), or banded (e.g., using elastic)) to a portion of the patient 140 such as the ear, fingertip, across a foot, or combinations thereof.

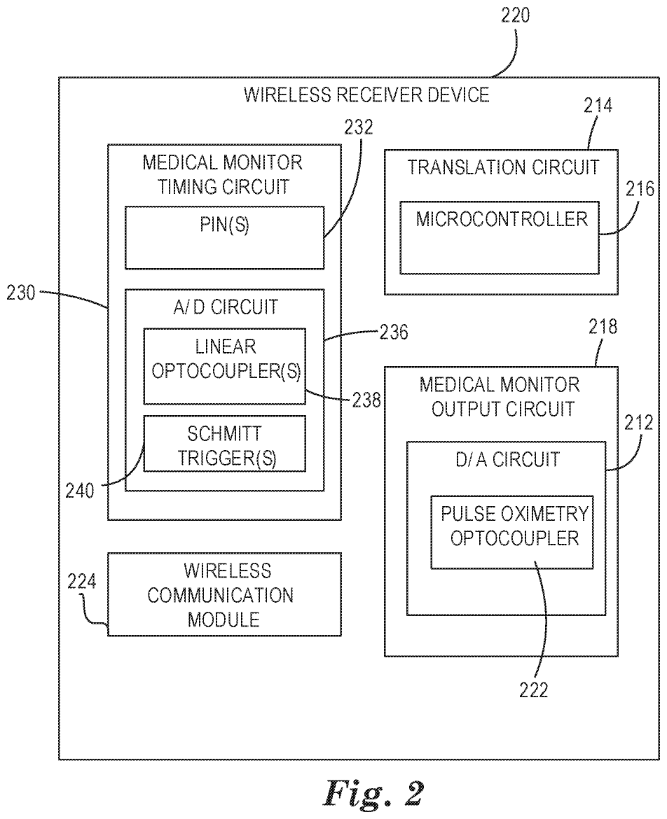

FIG. 2 illustrates a wireless receiver device 220. Wireless receiver device 220 can be similar to wireless receiver device 120. The wireless receiver device 220 interfaces between the pulse oximeter device 130 can the medical monitor 110. In at least one embodiment, the wireless receiver device 220 provides the medical monitor 110 with a photodiode signal sufficient for the medical monitor to construct a photoplethysmogram and determine the SpO2 (e.g., using internal processing).

The wireless receiver device 220 can have a medical monitor timing circuit 230, a wireless communication module 224, a translation circuit 214, and medical monitor output circuit 218 which are communicatively or electrically coupled to each other and each described further herein.

In at least one embodiment, the medical monitor timing circuit 230 can receive an LED activation signal from a medical monitor, and create a digital timing signal based on the LED activation signal. The LED activation signal can be a set of one or more analog currents with a particular amplitude and frequency. In at least one embodiment, the LED activation signal is a digital signal.

In at least one embodiment, the medical monitor timing circuit 230 can have one or more pins 232. The one or more pins 232 can form an electrical coupling with the medical monitor to receive at least one LED activation signal from the medical monitor. The one or more pins 232 can include a first pin and a second pin. In at least one embodiment, the first pin can be for reading at least one LED activation signal from a medical monitor, preferably, the first LED activation signal. The second pin can be for receiving the second LED activation signal. In at least one embodiment, the two pins can be used to determine if current is present and the direction of the current. In one example, the medical monitor timing circuit 230 can be configured to mate with DB9 ports.

In at least one embodiment, the medical monitor timing circuit 230 can have an analog to digital (A/D) circuit 236 for converting an analog current from the medical monitor to a digital timing signal. Ordinary artisans can construct the A/D circuit 236 in a variety of ways. However, one example of a construction of the AID circuit 236 can include one or more linear optocouplers 238 and one or more Schmitt triggers 240. It was found that inclusion of a linear optocoupler 238 and Schmitt trigger can perform further signal conditioning of the LED activation signal. The one or more linear optocouplers 238 can be electrically coupled to the a first medical monitor pin and/or a second medical monitor pin. In one example, the linear optocouplers 236 can be designed such that transmission wires are shared. In at least one embodiment, the Schmitt trigger 240 is electrically coupled to the one or more linear optocouplers and the translation circuit 214.

The linear optocouplers 238 can have a number of benefits. For example, a medical monitor 110 drive circuit can be configured to drive an LED. In addition to isolating the receiver device 220 from the medical monitor 110, the linear optocoupler 238 can be used to measure the magnitude of the LED activation signal current and can help determine whether the medical monitor 110 requires more or less optical signal returned on a photodiode return signal.

In at least one embodiment, the wireless receiver device 220 can include a wireless communication module 224. Examples of a wireless communication modules used throughout this disclosure are commercially available under the trade designation ATWINC 1500 from Microchip Inc. (Arizona, USA). The wireless communication module 224 can be configured for receiving a wireless signal sufficient to construct a photoplethysmogram and is described further herein. For example, the wireless signal can be a wireless photodiode signal, preferably, a portion of a wireless photodiode signal that corresponds to a portion of a photodiode response to received wavelength of light from an LED of the pulse oximeter. The wireless communication module 224 can be configured for receiving and/or transmitting the wireless signal. For example, the wireless communication module 224 can receive an R-value signal and/or the wireless photodiode signal as described herein and send a receipt confirmation.

In at least one embodiment, one or more wireless photodiode signals and the R-value signal can be received at different rates. For example, the wireless communication module 224 can receive the first wireless photodiode signal at a first rate and the R-value signal at a second rate.

In at least one embodiment, the wireless receiver device 220 can also include a translation circuit 214. The translation circuit 214 can be one or more circuits configured to determine an photodiode signal from the wireless signal based on a timing pulse of the at least one LED activation signal. For example, the first photodiode signal can be based on the timing of the first LED activation signal and the second photodiode signal can be based on the timing of the second LED activation signal. In at least one embodiment, the timing can be a digital timing signal.

In at least one embodiment, the photodiode signal can include one or more alternating values of first LED photodiode responses and second LED photodiode responses.

In at least one embodiment, the communications within the translation circuit 214 can be digital. For example, the input from the medical monitor timing circuit 230 to the translation circuit 214 can be digital. Also, the output to the medical monitor output circuit 218 can be digital.

In at least one embodiment, the translation circuit 214 can also include a microcontroller 216. Exemplary microcontrollers are commercially available under the trade designation STM32 from STMicroelectronics (Switzerland) or SAM C from Microchip Inc. (Arizona, USA). Generally, the microcontroller 216 can have one or more processors configured to control the timing of an photodiode signal. In at least one embodiment, one or more processors are configured to spool the first photodiode signal based on the first LED activation signal.

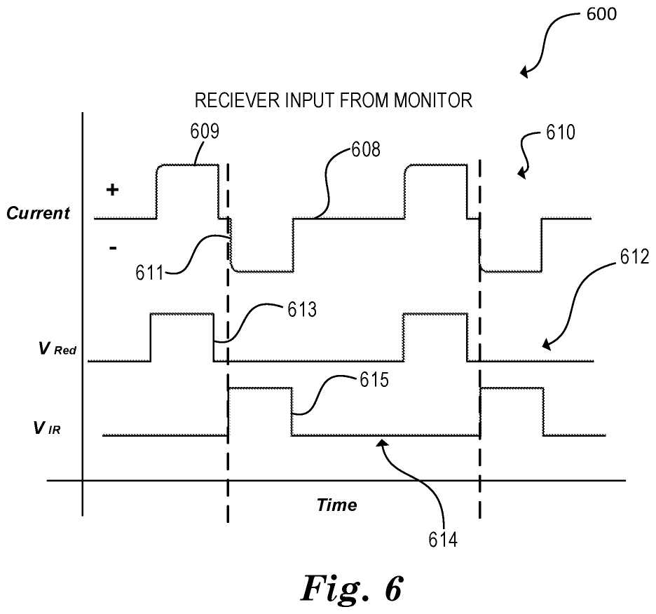

The wireless receiver device 220 can also include a medical monitor output circuit 218. The medical monitor output circuit 218 can be configured to provide an photodiode signal to the medical monitor. In at least one embodiment, the medical monitor output circuit 218 can be configured to convert digital signal to analog signals. For example, the medical monitor output circuit can receive an (first or second) photodiode signal and output an (first or second) photodiode signal current to the medical monitor 110. In at least one embodiment, a third photodiode signal can be provided when there is neither a first LED activation signal nor a second LED activation signal. Although mentioned as two signals, the LED activation signal current and the photodiode signal current can be continuous with varying values for the first photodiode signal current and the second photodiode signal current similar to that shown in FIG. 6.

In at least one embodiment, the medical monitor output circuit 218 can include a digital to analog (D/A) circuit 212. The D/A circuit 212 can be configured to covert the photodiode signal into a compatible input for the medical monitor (i.e., an photodiode signal current). For example, the D/A circuit 212 is configured to convert the first (or second) photodiode signal to a first (or second) photodiode signal current. The first or second LED photodiode current can be an analog signal current and can be provided to the medical monitor.

The D/A circuit 212 can include an optional pulse oximeter optocoupler 222. The pulse oximetry optocoupler 222 can provide an analog signal current within the range that is capable of being analyzed by a medical monitor. In at least one embodiment, the pulse oximetry optocoupler comprises components of a pulse oximeter. For example, the pulse oximetry optocoupler can include an LED and a photodiode. The LED can be any wavelength of light but is preferably red or IR as described herein. The LED can be configured to be compatible with the photodiode such that the photodiode is not oversaturated. The photodiode can also be compatible with the medical monitor. In at least one embodiment, the pulse oximetry optocoupler can include a cover to prevent external light interference. An example of an optocoupler that can be used as a pulse oximeter optocoupler or a linear optocoupler is commercially available under the trade designation IL300 from Vishay Intertechnology (USA).

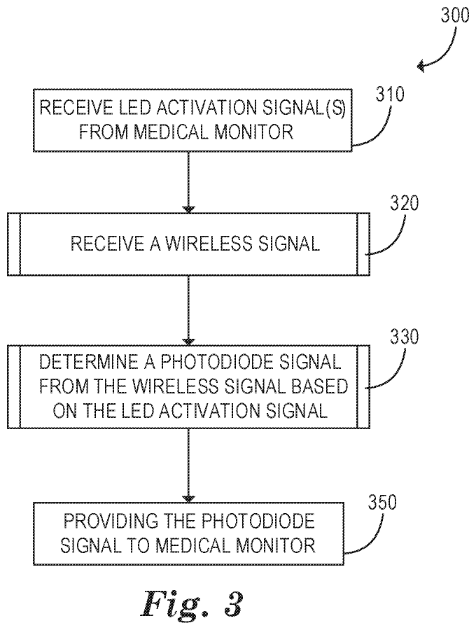

FIG. 3 illustrates a flowchart of a method 300 used by the wireless receiver device 220, according to at least one embodiment. Various aspects of the method 300 can be performed by components of the wireless receiver device 220. In at least one embodiment, method 300 can be order indeterminate, e.g., meaning that block 310 can take place after block 320.

The method 300 can begin at block 310. For example, the medical monitor timing circuit 230 can be configured to receive an LED activation signal from medical monitor 110 (i.e., through one or more pins 232). The LED activation signal can be an analog signal sufficient to activate an LED on a pulse oximeter. In various embodiments, the LED activation signal can have a different frequency of samples or sample rate than the wireless signal. The LED activation signal can also be conditioned, i.e., converted from an analog timing signal to a digital timing signal.

In block 320, the wireless communication module 223 can receive a wireless signal. As discussed herein, the wireless signal can be a digital signal with a specific timing. The wireless signal can be packet-based with various headers to indicate the proper destination of the data. Various encryption schemes can be used with the wireless signal such as 256-bit encryption. As part of receiving the wireless signal, the wireless communication module 223 can also extract a wireless photodiode signal and R-value signal which is described further herein. In another embodiment, extracting the photodiode signal can be performed by the translation circuit 214.

In block 330, the translation circuit 214 can determine an photodiode signal from the wireless signal based on the LED activation signal 330. In at least one embodiment, the photodiode signal can be a digital signal based on the digital timing of an LED activation signal. Block 330 can be described in further detail herein.

In block 350, the wireless receiver device 220 can provide the photodiode signal to the medical monitor. In at least one embodiment, the wireless receiver device 220 can output an analog photodiode signal current. Thus, a medical monitor output circuit 218 can be used to convert a digital signal of the photodiode signal to a photodiode signal current that is compatible with the medical monitor. In at least one embodiment, the medical monitor output circuit 218 can also use a pulse oximetry optocoupler comprising at least a photodiode that is compatible with the medical monitor.

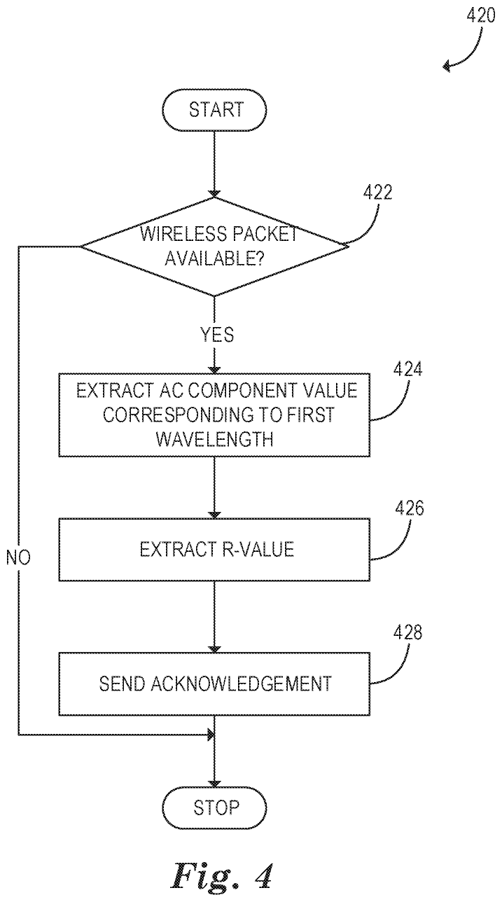

FIG. 4 illustrates a flowchart of a method 420 of receiving a wireless transmission. The method 420 can correspond to block 320 in FIG. 3. In at least one embodiment, the method 420 can occur for a packet-based wireless signal and different schemes may be used. In at least one embodiment, the extraction of different values may occur during block 330. The method 420 can begin at block 422.

In block 422, the wireless communication module 224 can receive the wireless signal and determine whether a network is present. If a wireless packet is available, then the method 420 can continue to block 424.

In block 424, the translation circuit 214 can extract an AC component value corresponding to a first wavelength of light. In at least one embodiment, the AC component value can be based on a rolling mean of AC component values as described further herein. In at least one embodiment, the AC component value can be a numeric value based on an photodiode response to an IR LED. The AC component value can be a segment of an IR photodiode signal. The extraction can occur based on one or more packet headers. The AC component is preferably bipolar due to the processing advantages during reconstruction of a photoplethysmogram, but can also be unipolar.

In block 426, the R-value, or R-value signal can be extracted from the wireless signal. As used throughout this disclosure, the R-value can be a numerical value. Although reference is made to R-value, the term R-value can also encompass measurements that are derived from the R-value. e.g., a SpO2 or perfusion index.

The R-value is a ratio of photodiode signals from each wavelength of light. In at least one embodiment, the R-value can correspond to the ratio of the a first wavelength of light arterial optical absorption to a second wavelength of light arterial optical absorption. In some embodiments, the R-value measures the ratio of the normalized derivative (or logarithm) of red intensity to the normalized derivative (or logarithm) of infrared intensity.

For example, as light passes through tissue, the light is scattered and absorbed by all the tissues, but the light passing through a pulsing artery or arterial bed will see a moving path length. The other tissues are unmoving and contribute to the steady non-pulsatile signal, but not to the time-varying pulsatile signals. The absorption of light by arterial blood is assumed to be only a function of the oxygenation state of the hemoglobin. Other basic assumptions are that the Red and InfraRed light travels along essentially the same optical path, and that the hardware circuits do not introduce any bias into the signal extraction.

The R-value can also indicate a change in the path length of distension with the blood pulse.

In block 428, the translation circuit 214 can instruct the wireless communication module 224 to send an acknowledgement to a sender that wireless packet was received.

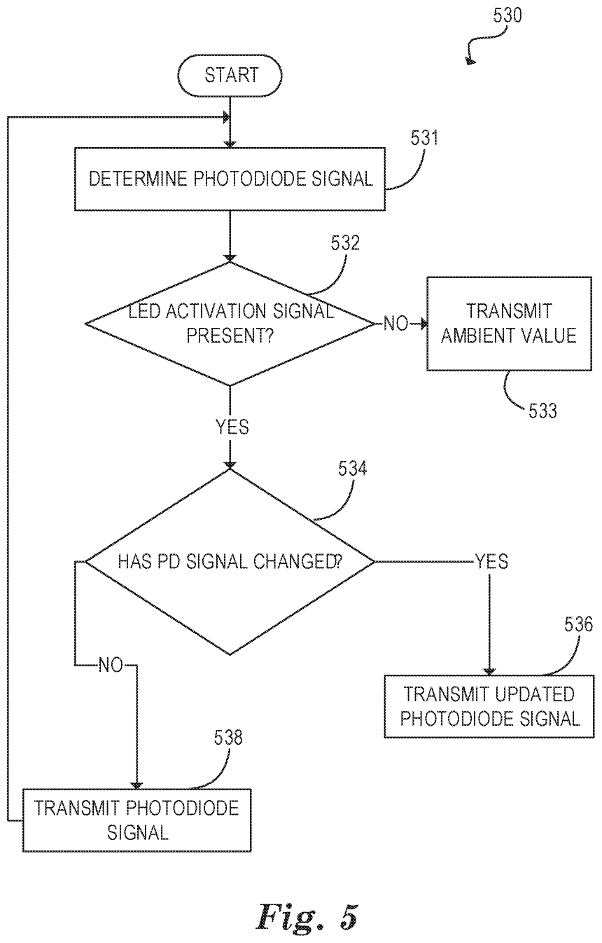

FIG. 5 illustrates a flowchart of a method 530 of determining an photodiode signal from the wireless transmission based on the LED activation signal. The method 530 can correspond to block 330 in FIG. 3. The method 530 can generally include timing the transmission of the photodiode signal based on a presence of an activation signal. The method 530 can begin at block 531.

In block 531, the translation circuit 214 can determine a photodiode signal based on the R-value and a wireless photodiode signal. In various embodiment, block 531 can occur concurrently with block 534 or before.

In at least one embodiment, the translation circuit 214 can determine an photodiode signal mathematically using the R-value and the AC component value. For example, the translation circuit 214 can determine a DC component offset value by receiving medical monitor parameters (e.g., which can be extracted from a magnitude of a medical monitors LED current pulse).

Throughout this disclosure, the DC component offset value can be the same or different than the DC component value determined on the pulse oximeter device. For example, a medical monitor may have a range of DC component offset values that are compatible with the medical monitor which may be different than the DC component value determined by the pulse oximeter device. The medical monitor parameters may be selected from model number, manufacturer, timing signal, LED current values (e.g., if the medical monitor needs more red PD signal, then more red LED current can be received), or any combination thereof.

The DC component offset value can be determined based on the medical monitor parameters. In at least one embodiment, the DC component offset value determination can also include selecting an arbitrary DC component offset value. Since the DC component offset value may be isolated by the medical monitor, then an accurate DC component offset value corresponding to a photodiode response to an LED though tissue is not necessary.

In at least one embodiment, the photodiode signal can be determined from a wireless photodiode signal and a first DC component offset value. For example, the determination can occur by receiving the AC component corresponding to a photodiode response to a first wavelength of light of a pulse oximeter, determining a first DC component offset value for the AC component, and adding the first DC component offset value to the AC component. For example, a value for the photodiode signal can be the sum of the first DC component offset value and the AC component for a particular wavelength of light.

The photodiode signal can be different when reconstructed from another wavelength of light. For example, a second photodiode signal can be determined partially from a second DC component offset value (which may be the same as the first DC component offset value) and the R-value. In at least one embodiment, a value from a second photodiode signal can be determined by receiving the AC component corresponding to a photodiode response from a first wavelength of light of a pulse oximeter (e.g., in block 426), determining a second DC component offset value for the AC component (which may be the same as the DC component offset value used for a first photodiode signal); and determining a product of the AC component and the R-value signal and adding the second DC component offset. For example, the IR AC component can be multiplied by the R-value to give a red LED AC component which can be added to a DC component to produce a value corresponding to a red photodiode signal.

In block 532, the translation circuit 214 can determine whether an LED activation signal is present. The LED activation signal can be present whenever the current is at a level sufficient to activate an LED (e.g., an IR or red LED). Since the LED activation signal can be digitalized by the medical monitor timing circuit, then the LED activation signal can be on or off. The LED activation signal can have three possible states. For example, a first LED active state, a second LED active state, and a dark or ambient state (off). If the LED activation signal is not present, then the method 530 continues to block 533.

In block 533, the translation circuit 214 can transmit an ambient value. The ambient value is a value from a photodiode corresponding to when both a first LED and second LED are not activated. The ambient value can refer to the general noise in the system due to light in the environment or heat produced from the patient. The ambient value can be estimated or a predetermined ambient value can be used. Generally, the ambient value transmitted can be zero. In at least one embodiment, a known offset value can be transmitted to simulate noise.

In block 534, the translation circuit 214 can determine whether the photodiode signal has changed. The photodiode signal has change whenever a value of a first or second wireless photodiode signal (and thus the photodiode value corresponding to a first or second wavelength) has changed. In at least one embodiment, the photodiode signal can change whenever the last received R-value changes.

In block 538, the translation circuit 214 can transmit an photodiode signal timed according to the LED activation signal 534. In various embodiments, the LED activation signal 534 can be converted to analog by determining an photodiode signal current and providing the photodiode signal current to the medical monitor.

In block 536, the translation circuit 214 can transmit the updated photodiode signal based on the new first or second wireless photodiode signal or the R-value.

FIG. 6 illustrates an exemplary timing diagram 600 of the medical monitor timing circuit 230. In at least one embodiment, the timing diagram 600 can be based off of timing from a FAST SpO2 module from an Intellivue MP2 made by Koninklijke Philips N. V., Netherlands. The timing diagram 600 can include a current measurement 610 corresponding to an LED activation signal originating from a patient monitor. The LED activation signal 610 can include a second LED activation signal 609 and a first LED activation signal 611, and third LED activation signal 608 corresponding to an ambient value or a “dark” signal. For example, the second LED activation signal 609 can correspond to a red LED and a positive current reading whereas the first LED activation signal 611 can correspond to a negative current reading. There may be a time gap between the first LED signal 611 and the second LED activation signal 609 such that a medical monitor timing circuit 230 can differentiate current pulses.

The medical monitor timing circuit 230 can convert the analog LED activation signal 610 into a digital activation signal 612. In at least one embodiment, the first or second LED activation signal 612, 614 (digital) can include one or more digital timing pulses (which may also be referred to as a digital timing signals. e.g., 613, 615). Each digital timing pulse can correspond to an analog LED activation signal. For example, the second LED activation signal (i.e., the pulse) 613 can correspond to second LED activation signal 609 and the first LED activation signal 615 can correspond to the first LED activation signal 611.

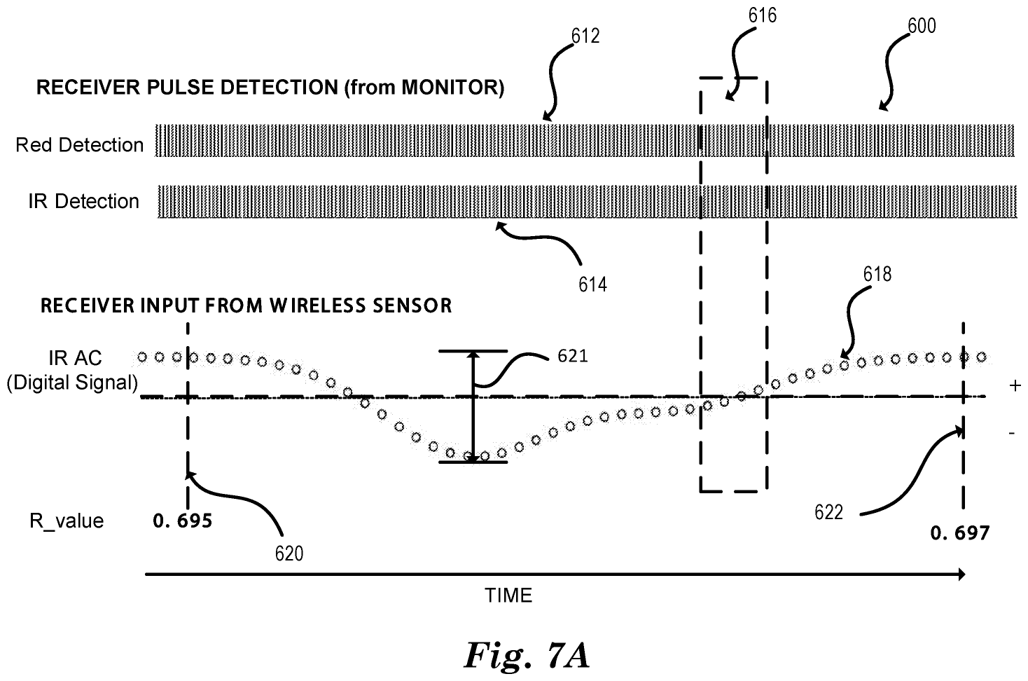

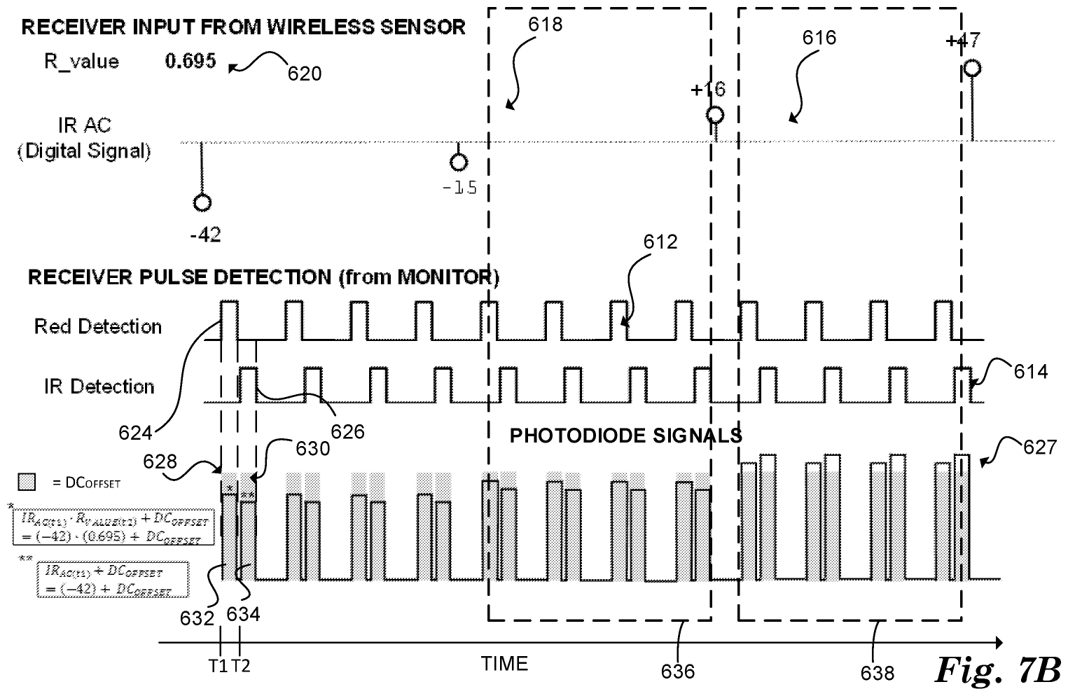

FIG. 7A-7B illustrate an aspects of the digital timing signals compared with values received from a wireless communication module.

In FIG. 7A, the first LED activation signal 614 and the second LED activation signal 612 are shown with less detail than in FIG. 6. Also shown with approximate time scale is a first wireless photodiode signal 618. The wireless photodiode signal 618 can be a set of values. In at least one embodiment, the wireless photodiode signal 618 can correspond to photodiode electrical responses to a first LED (e.g., an IR LED). Further, the wireless photodiode signal 618 can be filtered and correspond to an AC component value from the photodiode electrical response to an IR LED. As shown, the AC component value can be a signed integer value.

The wireless photodiode signal 618 can have values that occur in different sampling rates than the LED activation signals 614 and 612. As shown, the sampling rate of the first LED activation signal 614 is higher than the sampling rate of the wireless photodiode signal 618.

In addition to the wireless photodiode signal 618. R-values can also be received by the wireless communication module. The R-values 620, 622 can be received at a much lower sampling rate than either the wireless photodiode signal 618 or the LED activation signals 612, 614. Between R-values 620 and 622, the last received R-value can be utilized in any determinations of an photodiode signal. 621 illustrates a minimum and maximum difference.

In FIG. 7B, the area 616 is shown in greater detail to exemplify the processing of the translation circuit. Area 616 includes the wireless photodiode signal 618, first LED activation signal (digital) 614 and second LED activation signal (digital) 612.

The wireless photodiode signal 618 can include a −42 AC component value and an R-value of 0.695. The translation circuit 214 can use a receiver pulse detection signal (i.e., the first LED activation signal 614 and the second LED activation signal 612) to time the photodiode signal 627. The photodiode signal 627 can include values from both the first photodiode signal (e.g., 630) and the second photodiode signal (e.g., 628).

The first photodiode signal can include one or more values (e.g., 634) representative of a combined DC component and AC component of a photodiode electrical response to the first LED. The second photodiode signal can include one or more values (e.g., 632) representative of a combined DC component and AC component of a photodiode electrical response to the second LED.

The translation circuit can base the timing of values of the photodiode signal 627 on the LED activation signal. For example, value 632 can be time off the pulse 624 at T1 and the value 634 can be timed off of the pulse 626 at T2.

The value 632 can be determined using the exemplary calculations in *. The value 634 can be determined using the exemplary calculations in **. In at least one embodiment, the remainder of the photodiode signal 627 can be determined using the described calculations. The DC offset value used can be the same for both value 632 and 634 as described herein.

Region 636 and region 639 illustrate a concept in 534 FIG. 5. For example, in region 636, the last IR AC value was −15 and the last R-value was 0.695. Since the LED activation signals 612 and 614 have a higher frequency, then the values −15 and 0.695 can be used to determine 4 photodiode signals.

Similarly, the region 638 shows an update of the IR AC value of +16 but no change in the R-value. Thus, the next 4 photodiode signals can be identical and based on the timing of 612, 614.

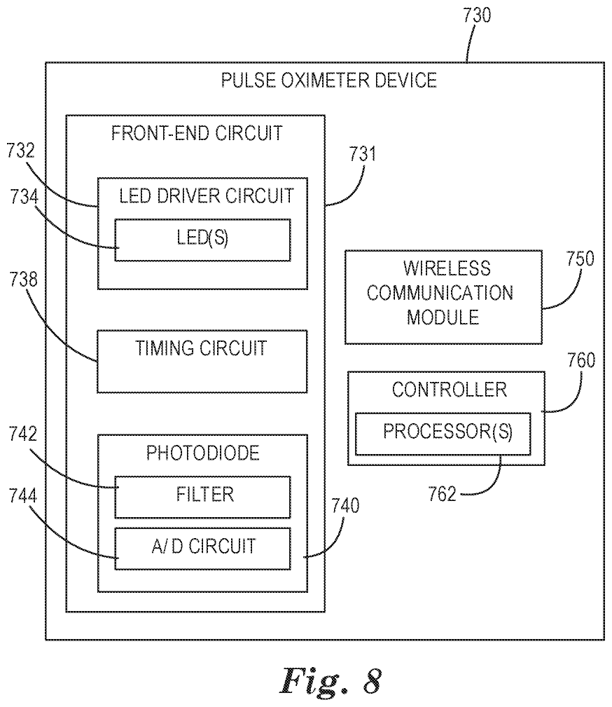

FIG. 8 illustrates a pulse oximeter device 730. The pulse oximeter device 730 can correspond to pulse oximeter device 130 in FIG. 1.

The pulse oximeter device 730 can have a front-end circuit 731. The front-end circuit can include an LED driver circuit 732, timing circuit 738, a photodiode 740. The front-end circuit can be for recording an electrical response to at least two wavelengths of light through a portion of a patient. Front-end circuits are known in the art and can be commercially obtained under the Model Numbers AFE4400 and AFE 4900 from Texas Instruments Inc., (Texas, USA).

The LED driver circuit 732 can be for controlling the timing and intensity of the LEDs (i.e., when the LEDs activate and deactivate). One aspect of the present disclosure is that a timing of the LEDs in the pulse oximeter device 730 is independent from a timing signal of a medical monitor 110 (and/or the wireless receiver device 120).

The LED driver circuit 732 can include one or more LEDs 734 (e.g., a first LED and/or a second LED). The one or more LEDs 734 can produce at least two wavelengths of light. For example, a first LED can produce a first wavelength of light and a second LED can produce a second wavelength of light. In at least one embodiment, the first LED can also produce a first and a second wavelength of light and paired with multiple photodiodes. The terms first LED and second LED can used throughout the disclosure to refer to either an IR LED or a red LED. Other wavelengths of LEDs can be used as will be appreciated by those of skill in the art. For example, green wavelength LEDs can also be used. In at least one embodiment, the first LED and the second LED can be an IR LED and a red LED, respectively. In some embodiments of the present disclosure, the first LED can be a red LED and the second LED can be an IR LED.

The first wavelength (e.g., IR) can be between 800 nm and 1100 nm (inclusive), or between 800 nm and 940 nm (inclusive). A second wavelength (e.g., red) can be between 600 and 800 nm (inclusive), or between 660 and 800 nm (inclusive). The first and second wavelength can differ by at least 1 nm.

The LED driver circuit 732 can also include a timing circuit 738 configured to provide a first LED activation current for the first LED and a second LED activation current for the second LED. As used throughout the disclosure, the term LED activation current can be similar to the LED activation signal current used throughout the specification except the LED activation current may have different timing from the LED activation signal current of the wireless receiver device.

The timing of the timing circuit 738 can be generated locally and is not dependent on a medical monitor. In at least one embodiment, the timing circuit 738 can vary the timing locally to optimize the photodiode readings and reduce noise. In some embodiments, the timing circuit 738 can be configured to mimic the timing and current of a medical monitor.

The pulse oximeter device 730 can have a photodiode 740 that can receive at least two wavelengths of light from at least one LED 735 that is transmitted through a portion of the patient 140. The at least two photodiode readings is indicative of light absorption of arterial blood in a patient at each of the at least two wavelengths of light. The photodiode 740 interacts with a pulsed LED to produce an electrical response that varies based on light transmittance through the patient 140. The variance of light absorption can reveal the oxygen absorption levels of the patient 140. Generally, the photodiode 740 is positioned facing the patient 140 and/or the LEDs.

The photodiode 740 can have an analog-to-digital circuit 744 and a filter 742. The filter 742 can filter ambient values (described further herein) from the photodiode readings.

The pulse oximeter device 730 can include a controller 760. In at least one embodiment, the controller 760 can coordinate red LED and IR LED pulses sufficient to measure oxygen content in blood. For example, the controller 760 can be electrically coupled to the photodiode 740 and the LED driver circuit 732. The controller 760 can control the LED driver circuit 732 and receive an photodiode reading from the photodiode 740. The controller 760 can have one or more processors 762 configured to extract an AC and a DC component from one or more photodiode readings and determine an R-value for a set of a plurality of photodiode readings. As used herein, the AC component can represent pulsatile arterial blood. The DC component can represent light absorption of the tissues, venous blood, and non-pulsatile arterial blood.

The controller 760 can also be coupled to the wireless communication module 750. The controller 760 can instruct the wireless communication module 750 to transmit only a portion of the photodiode reading from the photodiode 740. For example, the wireless communication module 750 can transmit an AC component value corresponding to one wavelength of light (and not the DC component value), and an R-value for a set of photodiode readings. The wireless signal can generally be digital and normalized for a full scale wireless photoplethysmogram.

Preferably, only the AC component value of an IR LED, and the R-value are transmitted. A wireless receiver device 120 or medical monitor 110 can reconstruct not only a oxygen absorption level, but also a full scale photoplethysmogram waveform for each wavelength of light. By transmitting only a portion of the photodiode reading using asynchronous timing (i.e., the timing is independent from the medical monitor) with respect to a medical monitor 110, the pulse oximeter device 730 can conserve battery life with minimal loss of resolution.

In at least one embodiment, the wireless communication module 750 can be configured to transmit in accordance with a power management scheme. For example, the pulse oximeter device 730 can be configured to transmit a wireless signal in short bursts such that the wireless communication module 750 can be deactivated for a certain period of time (thus saving energy). In one example, the wireless communication module 750 can be configured to transmit a series of wireless packets in short succession and deactivate for at least 100 ms.

The wireless communication module 750 can communicate with the wireless receiver device 120 using a variety of wireless signals 150 described herein.

In at least one embodiment, the wireless communication module 750 can be configured to transmit at least one wireless photodiode signal related to the photodiode response to transmitted light through a portion of a patient 140 from at least one LED. In at least one embodiment, the wireless photodiode signal can comprise one or more values, and a value can correspond to a the photodiode response to an LED. The at least one wireless photodiode signal can include a first wireless photodiode signal which is related to the photodiode reading of the first wavelength. In at least one embodiment, the first wireless photodiode signal comprises at least AC component value of a first photodiode reading of a first wavelength of light. In at least one embodiment, the first wireless photodiode signal comprises only the AC component. In at least one embodiment, the first wireless photodiode signal does not include the DC component but the DC component can be sent separate (optional)

In at least one embodiment, a second wireless photodiode signal can be optionally transmitted. The second wireless photodiode signal can include values that correspond to a photodiode response to a second LED.

The wireless communication module 750 can also be configured to transmit an R-value (e.g., a ratio of an optical absorption of a first wavelength of light to an optical absorption of a second wavelength of light, for a first set of photodiode readings). The R-value is described herein. In one example (which is only an example and is not limiting in any way), the R-value can be provided by the following equation:

R=(AC of 1st wavelength/DC of 1st wavelength)/(AC of 2nd wavelength/DC of 2nd wavelength) In at least one embodiment, the R-value can be determined from a rolling average of photodiode readings.

The R-value can be determined in a variety of ways. For example, the AC component values can be based partially on the min and max of a sample set of AC component values, a peak to peak analysis of the set of AC component values, or based on Root-mean-square of a plurality of AC component values. In at least one embodiment, the wireless communication module 750 can be configured to transmit a second wireless photodiode signal corresponding to received light from a second wavelength of the pulse oximeter device 730. For example, the second wireless photodiode signal can include an AC component value of the second wavelength or the AC component value and DC component value of the second wavelength.

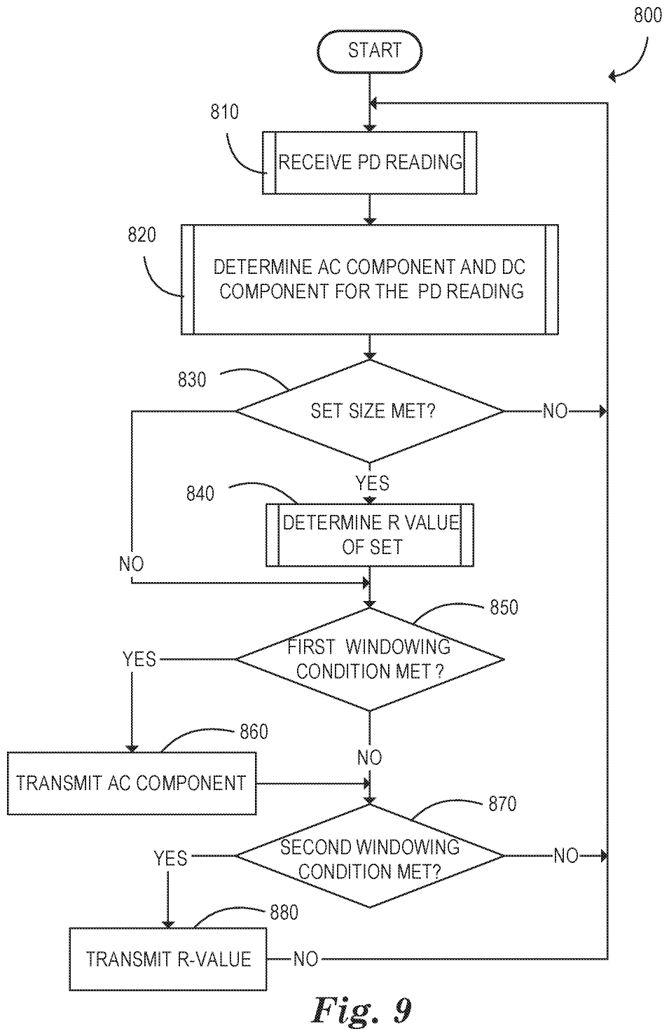

FIG. 9 illustrates a method 800 for processing one or more photodiode readings with the pulse oximeter device 730. The method 800 can begin in block 810.

In block 810, the controller 760 can be configured to receive an photodiode reading from the photodiode 740. As used herein, unless otherwise specified, reference to an photodiode reading can refer to either a photodiode reading from a first LED or a photodiode reading from a second LED. In at least one embodiment, approximately 150 to 500 samples per second can be obtained from the photodiode 740. Block 810 can be described further herein.

In block 820, the controller 760 can be configured to determine an AC component value and a DC component value for the received photodiode reading from block 810. The controller 760 can use one or more filters to determine the DC component value and then remove the value from a total value having both the DC component value and the AC component value. Block 820 can be described further herein.

In block 830, the controller 760 can be configured to determine whether a set size is met from one or more photodiode readings. The set size can be a number of photodiode readings that are analyzed (at least partially) as a set. In at least one embodiment, the set size is not static. For example, a first set can include some values that overlap with a second set. The set size is met if the number of values is at least a threshold number for a set. In at least one embodiment, the set size is at least two photodiode readings. The set size can also include enough samples for one complete blood pulse. For example, the range of blood pulses per second is 0.5 to 3 blood pulses/sec. In at least one embodiment, if the set size is not met, then the method 800 can continue to block 850. In at least one embodiment, if the set size is not met, then another photodiode reading can be obtained in block 810. If the set size is met, then the method 800 can continue to block 840.

In block 840, the controller 760 can be configured to determine an R-value for a first set of photodiode readings based on at least a normalized AC component value of the first set of photodiode readings. The R-value can be determined mathematically and is a ratio of two ratios, one ratio for each wavelength of light. The R-value determination can be described further herein.

In block 850, the controller 760 can be configured to determine whether a first windowing condition is met. The windowing condition defines the size and values encompassed by the window. The window may have overlapping values with the set size (set size used to perform the determination of the R-value in block 840). In at least one embodiment, the windowing condition can be related to the number of AC component values determined and may be lower than the number of photodiode readings. In at least one embodiment, the first windowing condition can be a count of AC component values for an wavelength of light.

In at least one embodiment, the first windowing condition can be a count of photodiode readings relative to AC component values for the wavelength of light. This may depend on how fast photodiode readings are taken. For example, the first windowing condition can range from 2 photodiode readings per 1 AC component value to 10 photodiode readings per 1 AC component value (inclusive) or 50 Hz.

In at least one embodiment, the first windowing condition can be a time condition for a set of photodiode readings. For example, the first windowing condition can be a time condition of 1-20 milliseconds(inclusive).

If the windowing condition is met, then the method 800 can continue to block 860. If the windowing condition is not met, then the method 800 can continue to block 870.

In block 860, the controller 760 can be configured to transmit the AC component value corresponding to at least one photodiode reading. The transmission can occur at various resolutions. For example, the AC component value can be transmitted between 8 bits and 24 bits. Transmission of a 16 bit value can offer a balance of precision (resolution on the receive side) and bandwidth. In at least one embodiment, a plurality of AC component values can be transmitted from 50 to 100 samples per second. In at least one embodiment, the AC component value for an LED can be normalized in a signed integer. In another embodiment, the AC component value can be transmitted as an unsigned integer. It was found that use of signed integers can unexpectedly improve processing performance despite the loss of one bit.

In block 870, the controller 760 can determine whether a second windowing condition is met. The second windowing condition can define a number of R-values. For example, the second windowing condition can be a time condition of 1 to 5 seconds. The second windowing condition can also be a count condition. In at least one embodiment, the second windowing condition can be based on the number of photodiode readings but it will depend on the sampling frequency of the sensor. For example, the second windowing condition can range from 2500 photodiode readings to 150 photodiode readings (per 1 R-value) (inclusive).

In at least one embodiment, the second windowing condition can be based on the number of AC component values received. For example, the second windowing condition can range from 50 AC component values to 500 AC component values (per 1 R-value) (inclusive). If the second windowing condition is met, then the method 500 can continue to block 880. If the second windowing condition is not met, then the method 500 can continue to block 810.

In block 880, the controller 760 can be configured to transmit (through the wireless communication module), the R-value for the first set of most recently computed photodiode readings. The transmission can be from 8 bits to 24 bits (inclusive) and be between 0.2 and 1 (inclusive) samples per second. In at least one embodiment, the R-value can be computed using a rolling computation where the R-value is updated with each sample.

In blocks 860 and 880, the transmission can further include a power management scheme. For example, a delay can be introduced such that a series of AC component values are packetized and the transmission is delayed. The delay of the transmission can allow the pulse oximeter device to temporarily deactivate to converse power. For example, the pulse oximeter device can have 100 milliseconds without a transmission, then 100 milliseconds of data included in each packet. The delay can be independent of the windowing in blocks 850 and 870.

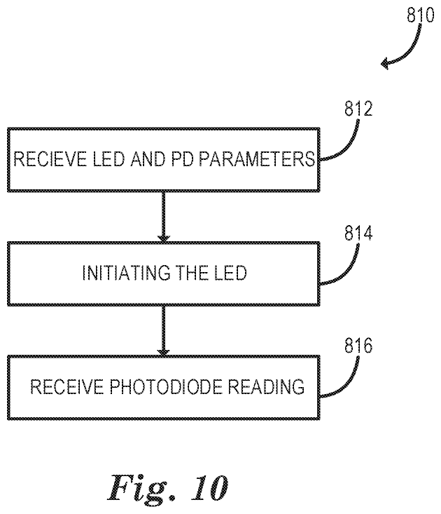

FIG. 10 illustrates block 810 in greater detail. Block 810 can include a method related to receiving a photodiode reading. Block 810 can begin by initiating the LED driver circuit 732.

In block 812, the controller 760 can initiate components of the pulse oximeter device 730. The pulse oximeter device components can each have one or more parameters that defines the operation of the pulse oximeter device 730. Examples of parameters include timing parameters, red LED parameters. IR LED parameters, and photodiode parameters. For example, the LED driver circuit 732 can have modifiable timing parameters, red LED parameters, and IR LED parameters. Thus, the intensity and timing of an LED activation current for an LED can be modified to produce an optimal photodiode reading. The photodiode 740 can also be adjusted to make the photodiode have adaptive gain toward light. Adjustments to the components in block 812 can occur before every photodiode reading within a set or after a certain number of readings (e.g., a set of photodiode readings). In some embodiments, the controller 760 can communicate the timing from the wireless receiver device to match the samples per second of the medical monitor.

In block 814, the controller 760 can instruct the LED driver circuit 732 to initiate an LED by providing an LED activation current to the first LED 736 or the second LED 734 according to the parameters in block 810.

In block 816, the controller 760 receive the photodiode reading from the photodiode 740. As discussed herein, the photodiode reading can correspond to an electrical response of the LED photodiode to either the first LED 736 or the second LED 734. In at least one embodiment, the photodiode 740 can also receive a third photodiode reading that corresponds to a dark phase where both the first LED and the second LED are off.

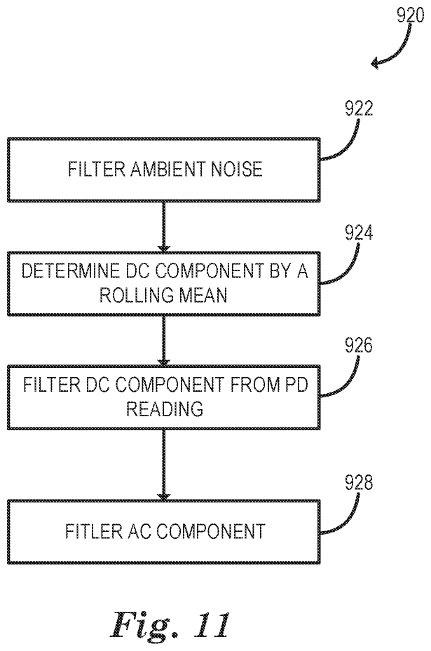

FIG. 11 illustrates a method 920 for determining AC component and DC component for the photodiode reading. Method 920 can be one embodiment of block 820 in FIG. 9 can include a method related to determining an AC component and DC component for an photodiode reading, method 920 can be performed by the controller 760.

In block 922, the controller 760 can be configured to filter ambient noise from the photodiode reading (forming a filtered photodiode reading). In at least one embodiment, the photodiode 740 can be coupled to a filter (e.g., 742) to perform the filtering. In at least one embodiment, an ambient reading (i.e., a third photodiode reading when both the first LED and second LED are deactivated) can be measured and used in the calculation of the photodiode signal (in the wireless receiver device). As used throughout this disclosure, the photodiode reading is preferably filtered but can also be unfiltered.

In block 924, the controller 760 can be configured to determine a DC component by a rolling mean of a plurality of first or second photodiode readings. In block 924, the rolling mean can be determined for photodiode readings corresponding to the first LED or the second LED. For example, if the 512th reading (e.g., corresponding to a rate of 500 samples per second) of a plurality of first photodiode readings (e.g., corresponding to an IR LED) is received, then the rolling mean can be measured from the 511 previous first photodiode readings to determine the rolling mean. Separately, if the 512^{th} reading of the plurality of second photodiode readings (e.g., corresponding to a red LED) is received, then the rolling mean can be measured from 511 second photodiode readings to determine the rolling mean.

The rolling mean can determine the DC component value. For example, an photodiode reading can have the AC component value as well as a DC component value, an averaged value can represent the DC component value (since the variable AC component represents the mean or average value over the last 512 samples within 1 second).

In block 926, the controller 760 can be configured to filter the DC component from an photodiode reading to obtain the AC component value. The AC component value can be the photodiode reading minus the DC component. In at least one embodiment, the count of the value determined in block 926 can be measured against a count condition of block 850.

In block 928, the controller 760 can be configured to optionally determine a rolling mean of the AC component value for an photodiode reading (which can filter high frequency noise above 50 Hz). In at least one embodiment, a rolling mean can be performed on the AC component value determined in block 926. For example, the rolling mean can be taken for the previous 10 AC component values for a first LED photodiode response. A rolling mean of the AC component values can serve to further balance out spikes that occur from the physical movement of the photodiode 740. The rolling mean of a set of AC component values can be transmitted in block 860. In at least one embodiment, the rolling mean determination of block 828 can be triggered based on the first windowing condition being met in block 850.

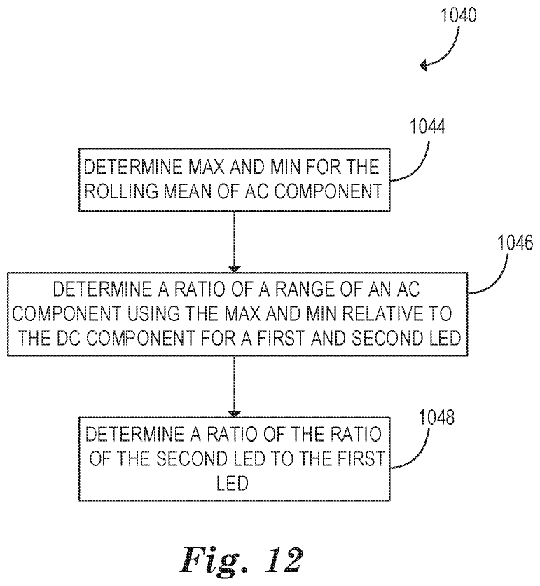

FIG. 12 illustrates a method 1040 of determining an R-value. Method 1040 can be an embodiment of block 840. Method 1040 can be performed by the controller 760.

In block 1044, the controller 760 can be configured to analyze a set of the AC component values to determine the local determine a maximum and minimum for the set of AC component values. For example, if 10 AC component values corresponding to a first LED are analyzed, and the maximum AC component value is 341 and the minimum AC component value is −171, then the controller 760 can note the values and determine a range of values. The range can be the difference between the maximum and minimum values. In the example above, the range can be 341−(−171)=512.

In block 1046, the controller 760 can be configured to determine a first LED photodiode ratio and a second LED photodiode ratio. The first LED photodiode ratio can be a ratio of the range of the AC component values (determined in block 1044) to the DC component value (determined in block 924) for the first LED. The second LED ratio can be a ratio of the range of the AC component values (determined in block 1044) to the DC component value (determined in block 924) for the second LED.

In block 1048, the controller can be configured to determine a ratio of the first LED photodiode ratio to the second LED photodiode ratio.

### List of Illustrative Examples

A1. A device, comprising:

a front-end circuit, comprising:
an LED driver circuit having at least one LED and configured to provide at least two wavelengths of light:
a photodiode configured to provide at least two photodiode readings to the at least two wavelengths of light from the at least one LED, wherein the at least two photodiode readings is indicative of light absorption of arterial blood in a patient at each of the at least two wavelengths of light.

a wireless communications module;

a controller, communicatively coupled to the front-end circuit and the wireless communication module, comprising:
one or more processors configured to:
   receive the at least two photodiode readings;
   determine an AC component value and a DC component value of a first one of the at least two photodiode readings;
   transmit the AC component value;
   determine an R-value, corresponding to a ratio of an optical absorption of a first wavelength of light to an optical absorption of a second wavelength of light, for a first set of photodiode readings;
   transmit the R-value for the first set of photodiode readings.
      A2. The device of Example A1, wherein the front-end circuit comprises a timing circuit configured to provide a first LED activation current and a second LED activation current for the at least one LED.
      A3. The device of Example A1 or Example A2, wherein one or more processors are configured to receive the at least two photodiode readings by:

initiating a first LED from the at least one LED by providing the first LED activation current sufficient to cause the first LED to emit a first wavelength of light;

receiving a first photodiode reading corresponding to an electrical response of the photodiode to the first wavelength of light.

A4. The device of Example A3, wherein the first LED is an IR LED.
A5. The device of Example A3, wherein the first LED is a red LED.
A6. The device of any of Examples A1 to A5, the wherein the LED driver circuit comprises a second LED configured to provide a second wavelength of light.
A7. The device of Example A6, wherein the second LED is a red LED.
A8. The device of any of Examples A1 to A7 wherein the front-end circuit comprises

a first LED configured to emit both a first wavelength of light and a second wavelength of light;

a first photodiode configured to receive the first wavelength of light;

a second photodiode configured to receive the second wavelength of light.

A9. The device of any of Examples A1 to A7, wherein the one or more processors are configured to determine whether a first set size is met from one or more photodiode readings.
A10. The device of Example A9, wherein the one or more processors are configured to

determine whether a first windowing condition is met in response to the first set size not being met.

A11. The device of Example A9, wherein the one or more processors are configured to:

determine a second photodiode reading in response to the first set size not being met.

A12. The device of any of Examples A9 to A11, wherein the one or more processors are configured to

determine the R-value in response to the first set size being met.

A13. The device of any of Examples A1 to A12, wherein the one or more processors are configured to transmit, wirelessly, an AC component value corresponding to at least one photodiode reading in response to a first windowing condition being met.
A14. The device of Example A13, wherein the first windowing condition is a count of photodiode readings relative to AC component values.
A15. The device of Example A14, wherein the first windowing condition ranges from 2 photodiode readings per 1 AC component value to 10 photodiode readings per 1 AC component value (inclusive).
A16. The device of any of Examples A13 to A15, wherein the first windowing condition comprises at least one blood pulse of a patient.
A17. The device of any of Examples A1 to A15, wherein the one or more processors are configured to transmit the R-value for the first set of photodiode readings in response to a second windowing condition being met.
A18. The device of Example A17, wherein the second windowing condition is a count of AC component values relative to R-values.
A19. The device of Example A17, wherein the second windowing condition ranges from 50 AC component values per 1 R-value to 500 1 AC component values per 1 R-value (inclusive).
A20. The device of Example A17, wherein the second windowing condition is a count of photodiode readings relative to R-values.
A21. The device of Example A20, wherein the second windowing condition ranges from 2500 photodiode readings per 1 R-value to 150 photodiode readings per 1 R-value (inclusive).
A22. The device of any of Examples A1 to A21, wherein the one or more processors are configured to cause the wireless communication module to transmit, wirelessly, the AC component value at at least 12 bits.
A23. The device of Example A20, wherein the one or more processors are configured to cause the wireless communication module to transmit, wirelessly, the AC component value as a signed integer.
A24. The device of any of Examples A1 to A22, wherein set of first photodiode readings is obtained at a first rate, the AC component value is determined at a second rate and the R-value is transmitted at a third rate.
A25. The device of Example A24, wherein the first rate is greater than the second rate.
A26. The device of Example A25, wherein the second rate is greater than the third rate.
A27. The device of any of Examples A24 to A26, wherein the first rate is 150 samples per second to 500 samples per second.
A28. The device of any of Examples A24 to A27, wherein the second rate is 50 to 100 samples per second.
A29. The device of any of Examples A24 to A28, wherein the third rate is 0.2 to 1 samples per second.
A30. The device of any of Examples A1 to A29, wherein the one or more processors are configured to determine an AC component value by:

filtering ambient noise from the photodiode reading to obtain a filtered photodiode reading;

determining a DC component; and

filtering the DC component from the filtered photodiode reading to obtain the AC component value.

A31. The device of Example A30, wherein the rolling mean is no greater than 100 photodiode readings.
A32. The device of any of Examples A30 or A31, wherein the one or more processors are configured to determine an R-value by:

determining a rolling mean of the AC component value for the first photodiode reading;

determining a range of the AC component value using a maximum and a minimum AC component value;

determining the optical absorption of the first wavelength of light based on the range of the AC component value and the DC component value:

determining the optical absorption of the second wavelength of light based on the range of the AC component value and the DC component value.

A33. The device of any of Examples A30 or A31, wherein the one or more processors are configured to determine the optical absorption of the first wavelength of light by determining a peak-to-peak value of a plurality of AC values.
A34. The device of any of Examples A30 or A31, wherein the one or more processors are configured to determine the optical absorption of the first wavelength of light by determining a root-mean-square of a plurality of AC values.
A35. The device of any of Examples A1 to A32, wherein the one or more processors are configured to receive a third photodiode reading when the at least one LED is off.
A36. The device of any of Examples A1 to A35, the front-end circuit comprises:

a first LED configured to emit a first wavelength of light and a second wavelength of light;

a second photodiode:

wherein a first photodiode is configured to receive the first wavelength of light, and the second photodiode is configured to receive the second wavelength of light.

A37. The device of any of Example A1 to A36, wherein the first wavelength of light ranges from 800 nm to 1100 nm (inclusive).
A38. The device of any of Examples A1 to A37, wherein the second wavelength of light ranges from 600 and 800 nm of light (inclusive).
A39. The device of any of Examples A1 to A38, wherein the first wavelength of light and the second wavelength of light differ by at least 1 nm.
A40. The device of any of Examples A1 to A39, wherein the R-value is a ratio between (i) a derivative of a plurality of first photodiode readings corresponding to light absorption in the patient from a first wavelength of light, and (ii) a derivative of a plurality of second photodiode readings corresponding to light absorption in the patient from a second wavelength of light.
A41. The device of any of Examples A1 to A40, wherein the derivative is a time derivative of the plurality of first photodiode readings is from at least one of a peak, a valley, or an average of at least one of the AC component to at least one of a peak, a valley, or an average of at least one of the red components.
A42. The device of any of Examples A1 to A41, wherein the optical absorption of a first wavelength of light is based partially on the AC component.
A43. The device of any of Examples A1 to A42, wherein the optical absorption of the first wavelength of light is based partially on the DC component.
B1. A method, comprising:

receiving an photodiode reading, wherein the photodiode reading is a first photodiode reading or a second photodiode reading:

determining an AC component value and a DC component value for the photodiode reading;

transmitting the AC component value corresponding to at least one photodiode reading;

determining an R-value for a first set of photodiode readings based on a normalized AC component value of the first set of photodiode readings:

transmitting the R-value for the first set of photodiode readings.

B2. The method of Example B1, wherein receiving a photodiode reading comprises:

initiating an LED by providing an LED activation current;

receiving the photodiode reading corresponding to an electrical response of the LED photodiode to the LED.

B3. The method of Example B2, wherein the LED comprises a first LED.
B4. The method of Example B3, wherein the first LED is an IR LED.
B5. The method of Example B2, wherein the LED comprises a second LED.
B6. The method of Example B5, wherein the LED is a red LED.
B7. The method of any of Examples B1 to B6, further comprising determining whether a first set size is met from one or more photodiode readings.
B5. The method of Example B7, further comprising determining whether a first windowing condition is met in response to the first set size not being met.
B9. The method of Example B7, further comprising determining another photodiode reading in response to the first set size not being met.
B10. The method of any of Examples B7 to B9, further comprising determining the R-value in response to the first set size being met.
B11. The method of any of Examples B1 to B10, wherein transmitting the AC component value corresponding to at least one photodiode reading occurs in response to a first windowing condition being met.
B12. The method of Example B11, wherein the first windowing condition is a count of photodiode readings relative to AC component values.
B13. The method of Example B11 TO B12, wherein the first windowing condition ranges from 2 photodiode readings per 1 AC component value to 10 photodiode readings per 1 AC component value (inclusive).
B14. The method of any of Examples B1 to B13, wherein transmitting the R-value for the first set of photodiode readings occurs in response to a second windowing condition being met.
B15. The method of Example B14, wherein the second windowing condition is a count of AC component values relative to R-values.
B16. The method of Example B15, wherein the second windowing condition ranges from 50 AC component values per 1 R-value to 500 1 AC component values per 1 R-value (inclusive).
B17. The method of Example B14, wherein the second windowing condition is a count of photodiode readings relative to R-values.
B18. The method of Example B17, wherein the second windowing condition ranges from 2500 photodiode readings per 1 R-value to 150 photodiode readings per 1 R-value (inclusive).
B19. The method of any of Examples B1 to B18, wherein transmitting the AC component value occurs at 16 bits.
B20. The method of any of Examples B7 to B19, wherein the set of first photodiode readings is obtained at a first rate, the AC component value is determined at a second rate and the R-value is transmitted at a third rate.
B21. The method of Example B20, wherein the first rate is greater than the second rate.
B22. The method of Example B20, wherein the second rate is greater than the third rate.
B23. The method of any of Examples B20 to B22, wherein the first rate is 150 samples per second to 500 samples per second.
B24. The method of any of Examples B20 to B23, wherein the second rate is 50 to 100 samples per second.
B25. The method of any of Examples B20 to B24, wherein the third rate is 0.2 to 1 samples per second.
B26. The method of any of Examples B1 to B25, wherein determine the AC component comprises:

filtering ambient noise from the photodiode reading to obtain a filtered photodiode reading;

determining a DC component value using a rolling mean; and

filtering the DC component value from the filtered photodiode reading to obtain the AC component.

B27. The method of Example B26, wherein the rolling mean is no greater than 512 photodiode readings.
B28. The method of any of Examples B26 or B27, wherein determine the AC component comprises:

determining a rolling mean of the AC component for the first photodiode reading;

determining a range of the AC component values using a maximum and a minimum AC component value;

determining a first LED photodiode ratio of the range of the AC component to the DC component value;

determining a second LED photodiode ratio of the range of the AC component to the DC component value; and

determining a ratio of the first LED photodiode ratio to the second LED photodiode ratio.

C1. A device comprising:

a front-end circuit, comprising:
an LED driver circuit having at least one LED and configured to provide at least two wavelengths of light;
a photodiode configured to provide at least two photodiode readings to the at least two wavelengths of light from the at least one LED, wherein the at least two photodiode readings is indicative of the light absorption of arterial blood at each of the at least two wavelengths of light.

a wireless communications module;

a controller, communicatively coupled to the front-end circuit and the wireless communication module, comprising:

one or more processors configured to perform the method of any of examples B1 to B28.

D1. A system comprising:

the device of any of Examples A1 to A43;

one or more sensors communicating with the device.

D2. The system of Example D1, wherein the one or more sensors are selected from a group consisting of: a blood pressure sensor, a biomedical electrode, a temperature sensor, and combinations thereof.
D3. The system of Example D1 or D2, wherein the system further comprises a medical monitor.
D4. The system of Example D3, wherein the medical monitor is configured to

receive one or more AC component values corresponding to the first wavelength of light and the R-value;

determine the photoplethysmogram for a patient from the one or more AC component values.

D5. The system of Example D3, wherein the medical monitor is configured to

receive a first photodiode signal current and a second photodiode signal current;

determine the photoplethysmogram for a patient based on the first photodiode signal current and the second photodiode signal current.

D6. The system of Example D5, wherein the medical monitor is configured to determine the photoplethysmogram by:

extracting an photodiode reading; and

displaying the photodiode reading.

D7. The system of Example D6, further comprising:

extracting the AC component value and the DC component value corresponding to a first wavelength of light and a second wavelength of light;

determining the R-value for the AC component values and the DC component values;

determining the SpO2 value from the R-value.

D8. The system of any of Examples D5 to D7, further comprising a wireless receiver device configured to:

receive a wireless signal from the wireless pulse oximeter device;

receive a first and second LED activation signal from the medical monitor;

provide a first and a second photodiode signal current to the medical monitor based on the first and second LED activation signal.

D9. The system of any of Examples D1 to D8, further comprising:

a patient, wherein the wireless pulse oximeter device is releasably attached to the patient.

E1. A device comprising:

a medical monitor timing circuit for reading at least one LED activation signal from a medical monitor;

a wireless communication module for receiving a wireless signal sufficient to construct at least one photoplethysmogram;

a translation circuit, communicatively coupled to the medical monitor timing circuit and the wireless communication module, for determining a first photodiode signal from the wireless signal based on a timing of the at least one LED activation signal; and

a medical monitor output circuit, communicatively coupled to the translation circuit, for providing the first photodiode signal to the medical monitor.

E2. The device of Example E1, wherein the translation circuit is configured to determine a second photodiode signal based on timing of a second LED activation signal.

E3. The device of Example E1 or E2, wherein the medical monitor output circuit further comprises:

a digital-to-analog conversion circuit configured to
convert the first photodiode signal to a first photodiode signal current, and
provide the first photodiode signal current to the medical monitor, wherein the first photodiode signal current is analog.
   E4. The device of Example E3, wherein the medical monitor output circuit provides a second photodiode signal current, based on a second photodiode signal, to the medical monitor.
   E5. The device of any of Examples E1 to E3, wherein the medical monitor timing circuit communicatively coupled to a medical monitor and configured to:

receive a first LED activation signal from a medical monitor, and

create a digital timing signal based on the first LED activation signal.

E6. The device of Example E5, wherein the medical monitor timing circuit is configured to:

receive a second LED activation signal from the medical monitor, and

create the digital timing signal based on the first LED activation signal and the second LED activation signal.

E7. The device of any of Examples E1 to E6, wherein the timing of the at least one LED activation signal is based on the digital timing signal.
E8. The device of any of Examples E1 to E7, wherein the translation circuit communicatively coupled to the medical monitor timing circuit and the wireless communication module comprising
one or more processors configured to:
   receive the digital timing signal from the medical monitor timing circuit,
   receive the wireless signal from the wireless communication module, and
   determine a first photodiode signal and a second photodiode signal based on the wireless signal and the digital timing signal.
      E9. The device of any of Examples E1 to E8, wherein the wireless signal comprises:

a first wireless photodiode signal corresponding to received light from at least two wavelengths of light from at least one LED of the pulse oximeter, and

a R-value signal, wherein an R-value is a ratio of an optical absorption of a first wavelength of light to an optical absorption of a second wavelength of light.

E10. The device of Example E9, wherein the first wireless photodiode signal corresponds to a portion of received light from an first LED of the pulse oximeter.
E11. The device of Example E10, wherein the first LED is an IR LED of the pulse oximeter.
E12. The device of Example E10, wherein the first LED emits a wavelength between 800 nm and 1100 nm (inclusive).
E13. The device of Example E12, wherein the first LED emits a wavelength between 800 nm and 940 nm (inclusive).
E14. The device of Example E10 or E11, wherein the first wireless photodiode signal comprises the AC component based on received light from the first LED that has been absorbed by a portion of a mammalian body.
E15. The device of Example E14, wherein the AC component represents pulsatile arterial blood.
E16. The device of Example E14 or E15, wherein the first wireless photodiode signal comprises the AC component and an DC component based on received light from the first LED that has been absorbed by a portion of a mammalian body.
E17. The device of Example E14 or E15, wherein the first wireless photodiode signal comprises only the AC component.
E18. The device of Example E14 or E15, wherein the first wireless photodiode signal does not include the DC component.
E19. The device of any of Examples E16 to E18, wherein the DC component represents light absorption of the tissues, venous blood, and non-pulsatile arterial blood.
E20. The device of Example E9, wherein the wireless signal comprises a second wireless photodiode signal corresponding to received light from a second LED of the pulse oximeter.
E21. The device of Example E20, wherein the second LED is a red LED.
E22. The device of Example E20 or E21, wherein the second LED emits a wavelength of between 600 and 800 nm (inclusive).
E23. The device of Example E20 or E21, wherein the second LED emits a wavelength of between 660 and 800 nm (inclusive).
E24. The device of any of Examples E1 to E23, wherein the one or more processors are configured to spool the first photodiode signal based on the first LED activation signal.
E25. The device of any of Examples E8 to E21, wherein the wireless communication module is configured to receive the wireless signal by:

receiving the first wireless photodiode signal at a first rate and the R-value signal at a second rate.

E26. The device of any of Examples E1 to E25, wherein the wireless signal is packet-based.
E27. The device of any of Examples E1 to E26, wherein the wireless signal is bit stream-based.
E28. The device of any of Examples E8 to E25, wherein the wireless communication module is configured to

transmit a confirmation of a receipt of the R-value signal and the first wireless photodiode signal.

E29. The device of any of Examples E8 to E28, wherein the wireless communication module is configured to transmit an offset value.
E30. The device of any of Examples E8 to E29, wherein the one or more processors are configured to determine the first photodiode signal by:

determining a first DC component offset value; and

determining the first photodiode signal from the first wireless photodiode signal and the first DC component offset value.

E31. The device of Example E30, wherein determining a DC component offset value comprises:

receiving medical monitor parameters;

determining the first DC component offset value based on the medical monitor parameters.

E32. The device of Example E31, wherein the medical monitor parameters are selected from a group consisting of: model number, manufacturer, timing signal, or any combination thereof.
E33. The device of Example E30, wherein determining the first DC component offset value comprises selecting an arbitrary DC component offset value.
E34. The device of Example E30 to E32, wherein the one or more processors are configured to determine the second wireless photodiode signal by:

determining a second DC component offset value; and

determining the second photodiode signal from the first wireless photodiode signal and the second DC component offset value.

E35. The device of Example E34, wherein the first DC component offset value is the same as the second DC component offset value.
E36. The device of any of Examples E8 to E35, wherein the one or more processors are configured to determine the second photodiode signal by:

determining the second photodiode signal from the first wireless photodiode signal, the DC component offset value, and the R-value signal.

E37. The device of any of Examples E1 to E36, wherein the medical monitor output circuit is communicatively coupled to the medical monitor and the translation circuit and configured to:

determine a first photodiode signal current from the first photodiode signal;

provide the first photodiode signal current to the medical monitor.

E38. The device of any of Examples E1 to E37, wherein the first photodiode signal is a digital signal based on a timing of the first LED activation signal.
E39. The device of any of Examples E1 to E38, wherein the first LED activation signal or the second LED activation signal is a set of analog current values with a particular amplitude and frequency.
E40. The device of any of Examples E1 to E39, wherein the first LED activation signal or the second LED activation signal is a digital signal.
E41. The device of any of Examples E1 to E39, wherein the first photodiode signal comprises a AC component and a DC component of a photodiode measurement from at least one LED of a pulse oximeter.

E42. The device of any of Examples E1 to E41, wherein the medical monitor timing circuit comprises:

one or more linear optocouplers communicatively coupled to the a first medical monitor pin for receiving the first LED activation signal and a second medical monitor pin for receiving the second LED activation signal;

a Schmitt trigger communicatively coupled to the one or more linear optocouplers and the translation circuit.

E43. The device of any of Examples E3 to E42, wherein the medical monitor output circuit comprises:

at least one linear opto-isolator, comprising:

an LED; and

a photodiode,

wherein the LED is equivalent to a red or IR LED from a pulse oximeter compatible with the medical monitor, and

wherein the photodiode is equivalent to a photodiode from the pulse oximeter compatible with the medical monitor.

E44. The device of any of Examples E3 to E43, wherein the first photodiode signal current is compatible with the medical monitor.
E45. The device of any of Examples E1 to E44, wherein the wireless signal is sufficient for a medical monitor to construct at least two photoplethysmograms.
E46. The device of any of Examples E1 to E45, wherein the device functions as a wireless receiver to a wireless sensor.
F1. A device comprising:

a translation circuit communicatively couple to a pulse oximetry photodiode and a medical monitor; the translation circuit comprising one or more processors configured to:
receive a first LED activation signal at a first time from a medical monitor,
receive a first wireless photodiode signal at a second time and an R-value at a third time;
determine a second photodiode signal based on the first wireless photodiode signal, the R-value, and the first LED activation signal;
determine the first photodiode signal current from a first photodiode signal and the second photodiode signal;
output the first photodiode signal current to the medical monitor.
   F2. The device of Example F1, wherein a difference between the second time and the first time is 10-20 ms.
   F3. The device of Example F1 or F2, wherein a difference between the first time and the third time is 1 to 5 seconds.
   F4. The device of any of Examples F1 to F3, wherein an LED activation signal, a wireless signal, or photodiode signal correspond to at least one value.
   F5. The device of any of Examples F1 to F4, wherein the LED activation signal corresponds to an on or off value.
   F6. The device of any of Examples F1 to F5, wherein a first wireless photodiode signal corresponds to at least one numeric value corresponding to a portion of a photodiode current reading from a first LED.
   F7. The device of any of Examples F1 to F6, wherein a second wireless photodiode signal corresponds to at least one numeric value corresponding to a portion of photodiode current reading from a second LED.
   F8. The device of any of Examples F1 to F7, wherein the R-value signal corresponds to at least one numeric value corresponding to a ratio of a first ratio of AC component to DC component for a first wavelength of light to a second ratio of an AC component to DC component for a second wavelength of light.
   F9. The device of any of Examples F1 to F8, wherein the LED activation signal, the wireless signal, or photodiode signal correspond to a plurality of values.
   G1. A device comprising:

one or more processors configured to:
receive at least one LED activation signal from the medical monitor at a first rate;
receive a wireless signal corresponding to an AC component and not a DC component of a photodiode response to light from a first LED at a second rate;
determining a first photodiode signal from the wireless signal based on the at least one LED activation signal; and
provide the first photodiode signal to the medical monitor at the first rate.
   H1. A method, comprising:

receiving at least one LED activation signal from a medical monitor;

receiving a wireless signal from a wireless pulse oximeter, wherein the wireless signal comprises at least an AC component of a photodiode response to light from a first LED of an pulse oximeter;

determining a first photodiode signal from the wireless signal based on the at least one LED activation signal; and

providing a first photodiode signal to a medical monitor.

H2. The method of Example H1, wherein receiving a wireless signal comprises:

determining if a wireless packet is available;

extracting, from the wireless packet, an AC component corresponding to a photodiode response to light from a first LED from a pulse oximeter;

extracting, from the wireless packet, an R-value corresponding to oxygen absorption in mammalian blood; and

sending acknowledgement of the wireless packet.

H3. The method of Example H1 or H2, wherein providing the first photodiode signal comprises:

determining whether a first LED activation signal is present;

determining whether the R-value or the AC component has changed:

transmitting a previous first photodiode signal in response to the R-value or the AC component not changing and the first LED activation signal being present.

H4. The method of Example H3, wherein determining whether a first LED activation signal is present comprises:

converting the first LED activation signal into a first LED activation signal; and

determining whether the first LED activation signal is present.

H5. The method of Example H3, wherein transmitting the first photodiode signal comprises:

converting the first photodiode signal to a first photodiode signal current;

transmitting the first photodiode signal current.

H6. The method of Example H1, wherein receiving at least one LED activation signal comprises:

receiving a first LED activation signal;

creating a digital timing signal based on the first LED activation signal.

H7. The method of Example H6, wherein receiving at least one LED activation signal comprises:

receiving a second LED activation signal from the medical monitor, and

create the digital timing signal based on the first LED activation signal and the second LED activation signal.

H8. The method of any of Examples H1 to H7, wherein determining a first photodiode signal comprises:

receiving the AC component corresponding to a photodiode response from a first wavelength of light;

determining a first DC component offset value for the AC component;

adding the first DC component offset value to the AC component.

H9. The method of any of Examples H1 to H8, further comprising:

determining a second photodiode signal from the wireless signal based on the at least one LED activation signal; and

providing a second photodiode signal to a medical monitor.

H10. The method of Example H9, wherein determining a second photodiode signal comprises:

receiving the AC component corresponding to at least one wavelength of light from at least one LED of a pulse oximeter;

determining a second DC component offset value for the AC component:

determining a product of the AC component and the R-value signal and adding the second DC component offset.

H11. The method of Example H9 or H10, wherein providing the second photodiode signal to a medical monitor comprises:

determining whether a second LED activation signal is present;

determining whether the R-value or the AC component has changed:

transmitting an updated second photodiode signal in response to the updated R-value or the AC component changing and the second LED activation signal being present.

H12. The method of any of Examples H1 to H11, further comprising:

determining whether a first or second LED activation signal is present;

providing an ambient value in response to the first or second LED activation signal not being present

H13. The method of Example H12, wherein the ambient value is a zero value.
H14. The method of Example H6, wherein the ambient value is a data state before the R-value or the AC component has changed.
I1. A system comprising:

the device of any of Examples E1 to E45.

I2. The system of Example I1, further comprising one or more sensors communicating with the device.
I3. The system of Example I2, wherein the one or more sensors are selected from a group consisting of: a blood pressure sensor, a biomedical electrode, a temperature sensor, and combinations thereof.
I4. The system of any of Examples I1 to I4, wherein the system further comprises a medical monitor.
I5. The system of Example I4, wherein the medical monitor is configured to receive a first photodiode signal current and a second photodiode signal current;

determine at least two photoplethysmograms for a patient based on the first photodiode signal current and the second photodiode signal current.

I6. The system of Example I5, wherein the medical monitor is configured to determine the photoplethysmogram by:

extracting an photodiode reading; and

displaying the photodiode reading.

I7. The system of Example I6, wherein the medical monitor is further configured to determine the photoplethysmogram by:

extracting the AC component value and the DC component value for the first LED and the second LED:

determining the R-value for the AC component values and the DC component values:

determining the SpO2 value from the R-value.

I8. The system of any of Examples I5 to I7, further comprising a wireless pulse oximeter device of any of Examples A1 to A43.
I9. The system of any of Examples 11 to 19, further comprising:

a patient, wherein the wireless pulse oximeter device is releasably attached to the patient.

J1. A system comprising:

a wireless pulse oximeter device configured to wirelessly transmit, based off of a first timing signal, (i) a plurality of AC component values corresponding to a first photodiode reading and not a DC component value, and (ii) an R-value, wherein the R-value is a ratio of at least two photodiode readings; and

a medical monitor configured to:
receive the plurality of AC component values and the R-value, and
determine a photoplethysmograph based on the plurality of AC component values and a second timing signal.
   J2. The system of Example J1, wherein the medical monitor is configured to determine an oxygen saturation value based on the R-value.
   J3. The system of Example J2, wherein the medical monitor is configured to display the oxygen saturation value.
   J4. The system of any of Examples J1 to J3, wherein the medical monitor is configured to display the photoplethysmograph.
   J5. The system of any of Examples J1 to J4, wherein the first timing signal and the second timing signal are different.
   J6. The system of any of Examples J1 to J5, further comprising:

a patient, wherein the wireless pulse oximeter device is releasably attached to the patient.

J7. The system of Example J6, wherein the wireless pulse oximeter device is attached to the ear, fingertip, esophagus, or chest of the patient.
J8. The system of any of Examples J1 to J7, wherein the wireless pulse oximeter device uses absorption of at least two wavelengths of light.
J9. The system of any of Examples J1 to J8, wherein the wireless pulse oximeter device uses a power management scheme.
J10. The system of Example J9, wherein the power management scheme deactivates a portion of the wireless pulse oximeter device and transmits a plurality of AC component values in bursts.
J11. The system of Example J10, wherein a deactivation time is at least 100 milliseconds.
J12. The system of any of Examples J1-J11, wherein the wireless pulse oximeter device is the device of any of Examples A1 to A43.
K1. A system, comprising:

a wireless pulse oximeter device having one or more processors communicatively coupled to a wireless communication module, the one or more processors are configured to wirelessly transmit an AC component value corresponding to a first photodiode reading from a biological source, and an R-value based off of a first timing signal; and

a medical monitor;

a wireless receiver device of any of Examples E1 to E45 communicatively coupled to the medical monitor.

K2. The system of Example K1, wherein the medical monitor has one or more processors configured to
provide an LED activation signal;
receive a first photodiode signal current and a second photodiode signal current;
determine the photoplethysmogram for a patient based on the first photodiode signal current and the second photodiode signal current.
   K3. The system of Example K2, wherein the medical monitor has one or more processors configured to determine the photoplethysmogram by:

extracting an photodiode reading; and

displaying the photodiode reading.

K4. The system of Example K3, further comprising:

extracting the AC component value and the DC component value corresponding to a first wavelength of light and a second wavelength of light:

determining the R-value from a set of AC component values and DC component values:

determining the SpO2 value from the R-value.

K5. The system of any of Examples K1 to K4, further comprising a wireless receiver device having one or more processors configured to:

receive a wireless signal from the wireless pulse oximeter device;

receive a first and second LED activation signal from the medical monitor;

provide a first and second photodiode signal current to the medical monitor based on the first and second LED activation signal.

K6. The system of Example K5, wherein the wireless receiver comprising a medical monitor output circuit communicatively coupled to the medical monitor, comprising:

a digital to analog conversion circuit comprising:
a pulse oximetry optocoupler comprising:
   a photodiode calibrated for use with the patient monitor.
      K7. The system of any of Examples K1 to K6, further comprising:

a patient, wherein the wireless pulse oximeter device is releasably attached to the patient.

K8. The system of any of Examples K1 to K7, wherein the wireless pulse oximeter is the device of any of Examples A1 to A43.

What is claimed is:

1. A device, comprising:
a front-end circuit, comprising:
an LED driver circuit having at least one LED and configured to provide at least two wavelengths of light;
a photodiode configured to provide at least two photodiode readings to the at least two wavelengths of light from the at least one LED, wherein the at least two photodiode readings is indicative of light absorption of arterial blood in a patient at each of the at least two wavelengths of light,
a wireless communications module;
a controller, communicatively coupled to the front-end circuit and the wireless communication module, comprising:
one or more processors configured to:
receive the at least two photodiode readings;
determine an AC component value and a DC component value of a first one of the at least two photodiode readings;
transmit the AC component value;
determine an R-value, corresponding to a ratio of an optical absorption of a first wavelength of light to an optical absorption of a second wavelength of light, for a first set of photodiode readings;
transmit the R-value for the first set of photodiode readings.

2. The device of claim 1, wherein the front-end circuit comprises a timing circuit configured to provide a first LED activation current and a second LED activation current for the at least one LED.

3. The device of claim 2, wherein one or more processors are configured to receive the at least two photodiode readings by:
initiating a first LED from the at least one LED by providing the first LED activation current sufficient to cause the first LED to emit a first wavelength of light;
receiving a first photodiode reading corresponding to an electrical response of the photodiode to the first wavelength of light.

4. The device of claim 3, wherein the first LED is an IR LED.

5. The device of claim 3, wherein the first LED is a red LED.

6. The device of any of claim 1, the wherein the LED driver circuit comprises a second LED configured to provide a second wavelength of light.

7. The device of claim 6, wherein the second LED is a red LED.

8. The device of any of claim 1, wherein the front-end circuit comprises
a first LED configured to emit both a first wavelength of light and a second wavelength of light;
a first photodiode configured to receive the first wavelength of light;
a second photodiode configured to receive the second wavelength of light.

9. The device of any of claim 1, wherein the one or more processors are configured to determine whether a first set size is met from one or more photodiode readings.

10. The device of claim 9, wherein the one or more processors are configured to
determine whether a first windowing condition is met in response to the first set size not being met.

11. The device of claim 9, wherein the one or more processors are configured to:
determine a second photodiode reading in response to the first set size not being met.

12. The device of claim 9, wherein the one or more processors are configured to
determine the R-value in response to the first set size being met.

13. The device of claim 1, wherein the one or more processors are configured to transmit, wirelessly, an AC component value corresponding to at least one photodiode reading in response to a first windowing condition being met.

14. The device of claim 13, wherein the first windowing condition is a count of photodiode readings relative to AC component values.

15. The device of claim 9, wherein the first set size is based off of at least one blood pulse of a patient.

16. A system comprising:
a wireless pulse oximeter device having one or more processors configured to wirelessly transmit, based off of a first timing signal, (i) a plurality of AC component values corresponding to a first photodiode reading and not a DC component value, and (ii) an R-value, wherein the R-value is a ratio of at least two photodiode readings; and
a medical monitor having one or more processors configured to:
receive the plurality of AC component values and the R-value, and
determine a photoplethysmograph based on the plurality of AC component values and a second timing signal.

17. The system of claim 16, wherein the medical monitor has one or more processors configured to determine an oxygen saturation value based on the R-value.

18. The system of claim 17, wherein the medical monitor comprises a display and is configured to display the oxygen saturation value.

19. The system of claim 18, wherein the medical monitor is configured to display the photoplethysmograph.

20. The system of claim 16, wherein the first timing signal and the second timing signal are different.
